(19) United States
(12) Patent Application Publication
(10) Pub. No.: US 2020/0126195 A1
(43) Pub. Date: Apr. 23, 2020

(54) IMAGING APPARATUS, METHOD, SYSTEM, AND STORAGE MEDIUM

(71) Applicant: Canon U.S.A., Inc., Melville, NY (US)

(72) Inventor: Badr Elmaanaoui, Belmont, MA (US)

(21) Appl. No.: 16/168,164

(22) Filed: Oct. 23, 2018

(51) Int. Cl. G06T 5/00; G06T 5/20; A61B 5/00

(52) U.S. Cl. CPC G06T 5/005; G06T 5/20; A61B 5/0082; A61B 2560/0223; G06T 2207/30101; G06T 2207/10101; A61B 2562/0233; A61B 5/0066

(57) ABSTRACT

An image processing apparatus includes a reference arm, a sample arm, a catheter, a catheter sheath, wherein the catheter is contained within the catheter sheath, and one or more processors to perform steps of: obtaining reflections from the catheter, and adjusting so as to place the catheter sheath a predetermined distance beyond a correct position in a manner where reflections from the catheter do not wrap and/or overlap a desired field of view.

### BACKGROUND

### Field of the Disclosure

The present disclosure generally relates to imaging and, more particularly, to an imaging apparatus, method, system, storage medium, and a technique for recovering wasted imaging range.

### Description of the Related Art

Imaging methodologies and technologies are implemented in a wide variety of areas to obtain a representation or reproduction of an object's form. Some areas include, for example, digital imaging, radar imaging, cinematography, photography, xerography, and the like. Medical imaging is an area that provides a technique and process of creating visual representations of the interior of a body for clinical analysis and medical intervention. Medical imaging technology is used to gain anatomic and physiologic data about a patient's body, organs, tissues, or a portion thereof for clinical diagnosis. Imaging applications may include imaging, evaluating, and diagnosing biological objects, such as, for example, gastro-intestinal, pulmonary, intravascular, and the like, and may be obtained via one or more instruments, such as, for example, one or more probes, one or more catheters, one or more endoscopes, one or more capsules, one or more needles, e.g. a biopsy needle, and the like.

Medical imaging can be used for both diagnosis and therapeutic purposes and common imaging types or modalities include transmission imaging, reflection imaging, and emission imaging. Medical imaging includes imaging types such as, for example, radiography (X-ray), fluoroscopy, confocal microscopy, computed tomography (CT), ultrasound, gray-scale/color Doppler, magnetic resonance imaging (MRI), positron emission tomography (PET), single-photon emission computerized tomography (SPECT), and the like. Imaging data may be obtained through non-invasive or invasive procedures. A wide variety of medical fields benefit from information obtained through medical imaging including, for example, ophthalmology, optometry, cardiology, neuroscience, oncology, orthopedics, and the like.

Intravascular imaging (IVI) modalities may provide cross-sectional imaging of coronary arteries with precise lesion information (e.g., lumen size, plaque morphology, implanted devices, and the like). In the U.S.A., roughly only 20% of interventional cardiologists currently use IVI imaging in conjunction with coronary angiography during percutaneous coronary intervention (PCI). Typical IVI configurations generally provide around a to mm imaging field of view (FOV) so as to be able to image vessels as large as 3.0 mm to 3.5 mm in diameter due to factors such as catheter eccentricity, oblique beam illumination and the desire to visualize tissue to a depth of at least 1.0 mm to 1.5 mm. However, a small percentage of vessels can be as large as 4.0 to 4.5 mm, especially closer to the ostia and in the left main coronary artery.

Optical coherence tomography (OCT) is an imaging technique that uses coherent light to capture micrometer resolution, two- and three-dimensional images from within optical scattering media. OCT may provide invasive, non-invasive, or minimally invasive three-dimensional (3D) imaging techniques with advantages over competing methods in axial and lateral resolution. OCT may synthesize a cross-sectional image (B-scan image) from a series of laterally adjacent depth-canvas (A-scans), and a 3D image of the sample can be constructed by recording multiple adjacent B-scans.

Ultrasound typically uses acoustic waves with frequencies ranging between around 3 to 40 MHz and gives a resolution of around 0.1 to 1 mm. High frequency ultrasound of around too MHz provides imaging resolutions of around 15 to 20 μm, and imaging depths are limited to only a few millimeters because biological tissues strongly attenuate the high frequency sound waves. Confocal microscopy has high resolution approaching 1 μm, but has a typical imaging depth of only a few hundredths of microns.

OCT technology may be compared to and is generally analogous to ultrasound but OCT technology uses light instead of sound. As the wavelength of light is much shorter than that of ultrasound, using light instead of ultrasound significantly improves image resolution. Ultrasound and OCT are analogous in that when a beam of sound or light is sent to a biological tissue, it is back-reflected or backscattered differently from structures that have varying acoustic or optical properties, as well as boundaries between structures. The dimensions of those structures can be determined by measuring the “echo” time it takes for sound or light wave to return from various axial distances. Because the speed of light is much faster than sound, a time resolution of around 50 femtoseconds takes place with OCT, and the small scale echo time delay measurement can be achieved using low-coherence interferometry. As such, OCT can be characterized as an interferometric-based imaging technique that can measure the amplitude and echo time delay of backscattered light with high sensitivity. In interferometric imaging, light from a reference path, e.g. a known and controlled optical path, is caused to interfere with light returned from a sample path, e.g. an unknown path, so information from the sample path can be determined by an analysis of the interferometric data.

OCT is useful in performing non-invasive or minimally invasive medical procedures based in part on OCT compatibility with fiber optics. In OCT applications, an interferometer may be used to split light into fiber optic-based sample and reference paths. The length of the reference path is normally adjusted to match the length of the sample path. The difference between the length of the sample and the length of the reference path is the z-offset, which is zero when the paths have matched lengths. If the z-offset is known, the system can be calibrated by changing the length of the reference path to match the length of the sample path.

In OCT applications, an optical zero-point is normally defined where a reference plane exists in an image space. Surface planes are in the x-y plane, and depth occurs along the z axis. OCT technology may provide an image resolution of around 1-15 μm and an imaging depth ranging from around 1 to 3 mm. Effects of absorption and scattering within biological samples can limit the imaging depth in OCT technology. Categories of OCT technology generally include time domain OCT (TD-OCT) and Fourier domain OCT (FD-OCT). The axial resolution of OCT technology may be determined by the bandwidth of the light source in that the broader the source bandwidth, the better the axial resolution.

TD-OCT is based on the principles of reflectance low-coherence interferometry. A typical TD-OCT configuration includes a two-beam interferometer, such as a Michelson interferometer, with source, reference, sample, and detection paths. Fiber based OCT may be used for portable and endoscopic (catheter) imaging applications. A sample is positioned in one arm and a mirror is positioned in a reference arm. A signal is acquired by translating the reference mirror continuously along the beam axis. TD-OCT and FD-OCT each generally use a reference arm and an interferometer to detect echo time delays of light. The interferometer uses a beamsplitter and divides the light into a target arm and a reference arm. The reference arm in TD-OCT is mechanically scanned by a moving mirror in order to produce a time-varying time delay. The light source in FD-OCT is frequency swept and the interference of the light beams oscillates according to the frequency difference.

FD-OCT includes spectral domain OCT (SD-OCT) and swept source OCT (SS-OCT). Depth information can be provided by computing the inverse Fourier Transform of the spectrum of the backscattered interfering light. Imaging speed is greatly improved in FD-OCT compared with TD-OCT since scanning of a reference mirror is not necessary. The spectrum of backscattered light can be obtained by a spectrometer based technique in the SD-OCT case or a wavelength tunable laser source in the case of SS-OCT. In a swept source OCT configuration, the spectrum of the backscattered light is encoded in time by serially and rapidly tuning the wavelength of laser emitting light. For equivalent signal-to-noise ratio (SNR), swept source OCT with balanced detection provides a higher acquisition rate compared with spectral domain FD-OCT. The image quality of an OCT imaging system may be characterized based on factors including, for example, axial and transverse resolution, SNR, sensitivity, and penetration depth.

OCT resolution includes axial and transverse resolution. Axial resolution may be determined by the power spectrum of the light source. Transverse resolution depends on the wavelength and sample arm imaging optics. There is a tradeoff between transverse resolution and depth of focus, where a system with a finer transverse resolution corresponds to a shorter depth of focus. The optical resolution can be described based on the axial direction (Z) and the lateral dimension (XY).

Image processing technology may be enhanced by overcoming or increasing limits of the depth range of imaging configurations.

### SUMMARY

The present disclosure generally achieves improvements in image processing technology, for example, by increasing the size of vessels that can be imaged without changing the acquisition electronics or catheter size.

According to an aspect of the present disclosure, an image processing apparatus includes a reference arm, a sample arm, a catheter, a catheter sheath, wherein the catheter is contained within the catheter sheath, and one or more processors is provided. The processer is configured to perform steps of: obtaining reflections from the catheter, and adjusting so as to place a reflection defining the catheter sheath a predetermined distance beyond a correct position in a manner where reflections from the catheter do not wrap and/or overlap a desired field of view.

According to other aspects of the present disclosure, an image processing method for an image processing apparatus is provided. The method comprising: obtaining reflections from a catheter; and adjusting so as to place a catheter sheath a predetermined distance beyond a correct position in a manner where reflections from the catheter do not wrap and/or overlap a desired field of view.

According to other aspects of the present disclosure, a storage medium storing a program for causing a computer to execute the image processing methods for an image processing apparatus as described herein.

Further features of the present disclosure will become apparent from the following description of exemplary embodiments with reference to the attached drawings.

### BRIEF DESCRIPTION OF THE DRAWINGS

FIG. 1(A) is a diagram showing an embodiment of an OCT system in accordance with one or more aspects of the present disclosure;

FIG. 1(B) is a diagram of an embodiment of a catheter that may be used with at least one embodiment of an OCT system in accordance with one or more aspects of the present disclosure;

FIG. 2 is a flow chart according to a first exemplary embodiment.

FIG. 3(A) illustrates a digital image representation of an image according to an exemplary embodiment.

FIG. 3(B) illustrates the digital image of FIG. 3(A) converted to polar representation according to an exemplary embodiment.

FIG. 4(A) illustrates a digital image representation of an image according to an exemplary embodiment.

FIG. 4(B) illustrates a digital image representation of an image according to an exemplary embodiment.

FIG. 5(A) illustrates a digital image representation of an image according to an exemplary embodiment.

FIG. 5(B) illustrates a digital image representation of an image according to an exemplary embodiment.

FIG. 6(A) illustrates a digital image representation of an image according to an exemplary embodiment.

FIG. 6(B) illustrates a digital image representation of an image according to an exemplary embodiment.

FIG. 7 shows a schematic diagram of an embodiment of a computer that may be used with the OCT system in accordance with one or more aspects of the present disclosure.

FIG. 8 shows a schematic diagram of an alternative embodiment of a computer that may be used with the OCT system in accordance with one or more aspects of the present disclosure.

### DESCRIPTION OF THE EMBODIMENTS

Exemplary embodiments of the present disclosure will be described below with reference to the drawings.

While the following illustrates exemplary embodiments of the present disclosure, the configurational aspects of the present disclosure may advantageously be applied to other image processing apparatus, method, system, and storage medium configurations, including, for example, transmission imaging, reflection imaging, and emission imaging including, all types of OCT and ultrasound configurations, radiography (X-ray), fluoroscopy, confocal microscopy, computed tomography (CT), ultrasound, gray-scale/color Doppler, magnetic resonance imaging (MRI), positron emission tomography (PET), single-photon emission computerized tomography (SPECT), and the like.

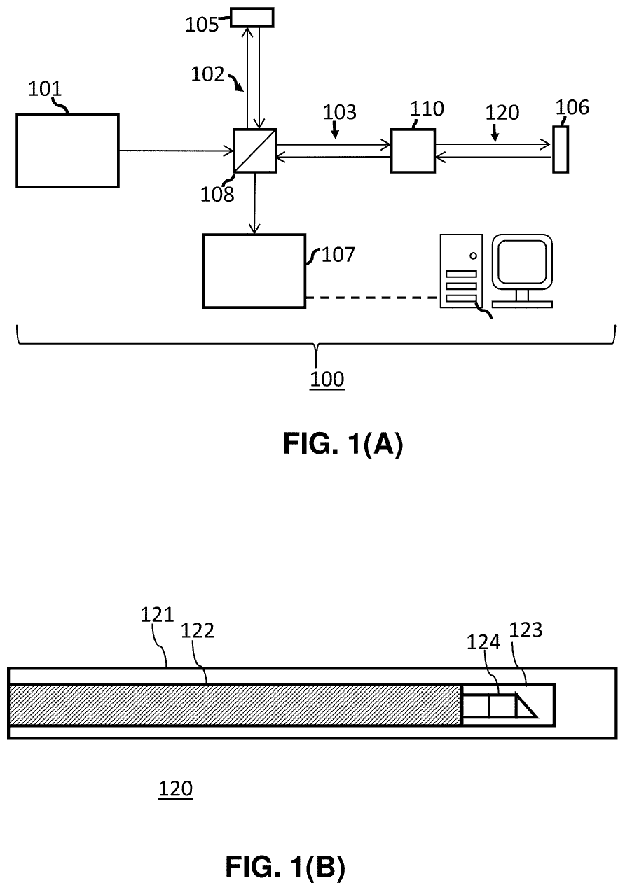

FIG. 1 illustrates an image processing apparatus configured as an optical coherence tomography (OCT) system 100 according to one or more aspects of the present application.

Turning now to the details of the figures, FIG. 1(A) shows the imaging processing apparatus 100 (as referred to herein as “system 100” or “the system 100”) which operates to utilize an OCT technique with optical probe applications in accordance with one or more aspects of the present disclosure. The system 100 comprises a light source lot, a reference arm 102, a sample arm 103, a deflected or deflecting section 108, a reference mirror (also referred to as a “reference reflection”, “reference reflector”, “partially reflecting mirror” and a “partial reflector”) 105, and one or more detectors 107. In one or more embodiments, the system 100 may include a patient interface device or unit (“PIU”) 110 and a catheter 120 (as diagrammatically shown in FIG. 1(A); an embodiment example is shown in FIG. 1(B) and discussed further below), and the system 100 may interact with a sample 106 (e.g., via the catheter 120 and/or the PIU 110). In one or more embodiments, the system 100 includes an interferometer or an interferometer is defined by one or more components of the system 100, such as, but not limited to, at least the light source lot, the reference arm 102, the sample arm 103, the deflecting section 108 and the reference mirror 105.

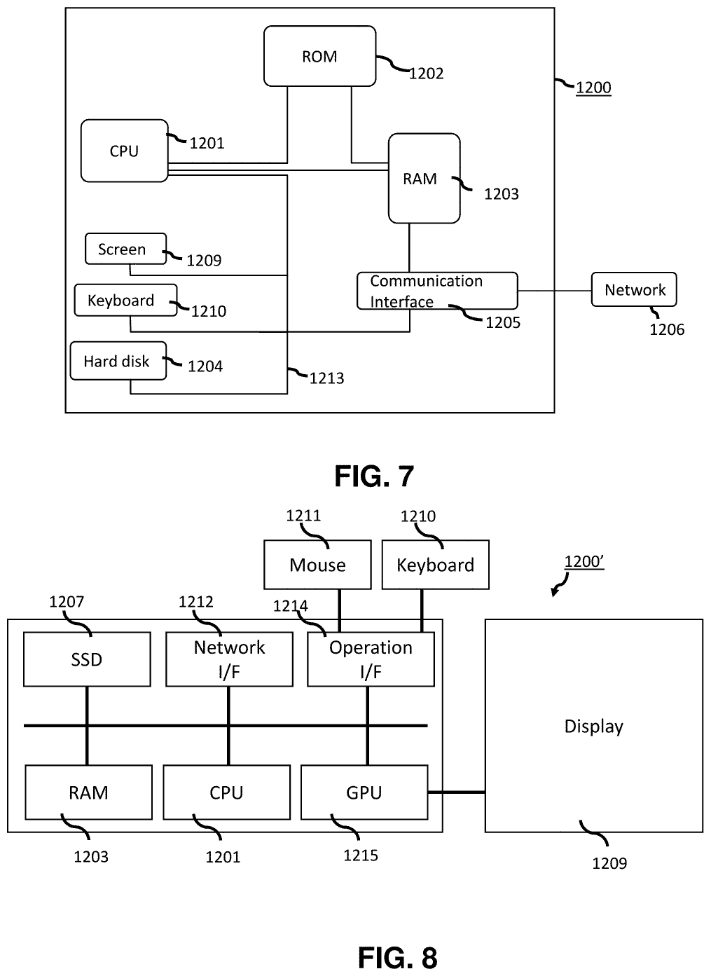

The light source lot, which may be, for example, a wavelength scanning light source, operates to produce a light to the deflecting section 108, which splits the light from the light source 101 into a reference beam passing into the reference arm 102 and a sample beam passing into the sample arm 103. The deflecting section 108 may be positioned or disposed at an angle to the reference mirror 105, the one or more detectors 107 and to the sample 106. The reference beam is reflected from the reference mirror 105 in the reference arm 102 while the sample beam is reflected or scattered from a sample 106 through the PIU (patient interface unit) 110 and the catheter 120 in the sample arm 103. Both of the reference and sample beams combine (or recombine) at the deflecting section 108 and generate interference patterns. The output of the system 100 and/or the interferometer thereof is continuously acquired with the one or more detectors 107, e.g., such as, but not limited to, photodiodes or multi-array cameras. The one or more detectors 107 measure the interference or interference patterns between the two radiation or light beams (e.g., a reference beam from the reference arm 102 and a sample beam from the sample arm 103) that are coupled, combined or recombined. In one or more embodiments, the reference and sample beams have traveled different optical path lengths such that a fringe effect is created and is measurable by the one or more detectors 107. Electrical analog signals obtained from the output of the system 100 and/or the interferometer thereof are converted to digital signals to be analyzed with a computer, such as, but not limited to, the computer 1200 (see FIG. 1(A); also shown in FIG. 7 discussed further below), the computer 1200′ (see e.g., FIG. 8 discussed further below), etc. In one or more embodiments, the light source 101 may be a radiation source or a broadband light source that radiates in a broad band of wavelengths. In one or more embodiments, a Fourier analyzer including software and electronics may be used to convert the electrical analog signals into an optical spectrum.

Preferably, the deflected section 108 operates to deflect (or split) the light from the light source 101 to the reference and sample arms 102, 103 to the reference mirror 105 and the sample 106, respectively, and then combine or recombine the light and send the combined or recombined light received from the reference mirror 105 and the sample 106 towards the at least one detector 107. In one or more embodiments, the deflected section 108 of the system 100 may include or may comprise one or more interferometers or optical interference systems that operate as described herein, including, but not limited to, a circulator, a beam splitter (see e.g., FIG. 1(A)), an isolator, a coupler (e.g., fusion fiber coupler), a partially severed mirror with holes therein, a partially severed mirror with a tap, etc. In one or more embodiments, the interferometer or the optical interference system may include one or more components of the system 100, such as, but not limited to, one or more of the light source lot, the reference arm 102, the sample arm 103, the deflected section 108 and/or the reference reflection 105. In one or more embodiments, the deflected or deflecting section 108 may include common path components, such as, but not limited to, a common path interferometer, a common path optical interference system, etc.

In one or more embodiments of an interferometer (e.g., a Michelson interferometer), a light source, such as the light source lot, operates to produce a light to a splitter, which splits the light from the light source 101 into a reference beam passing into a reference arm and a sample beam passing into a sample arm, which may be physically separate arms. In such an interferometer, a deflection section (such as the deflection section 108, which may be a beam splitter or other suitable component as described hereinabove) may be positioned or disposed at an angle to a reference mirror (such as the reference mirror 105), at least one detector (such as the detector 107) and to a sample (such as the sample 106). The reference beam is reflected from a reference mirror (such as the reference reflection 105) in the reference arm while the sample beam is reflected or scattered from a sample (such as the sample 106) in the sample arm.

In one or more embodiments, the reference reflector or reference reflection 105 may be disposed in the system 100 at least one of: (i) at the start of the image field of view (FOV) or at a proximal edge of imaging; and (iii) at a position that is shorter than a predetermined or desired start of the image FOV.

One application of an OCT technique of the present disclosure is to use the technique with a catheter 120 as schematically shown in FIG. 1(A). FIG. 1(B) shows an embodiment of the catheter 120 including a sheath 121, a coil 122, a protector 123 and an optical probe 124. As shown schematically in FIG. 1(A), the catheter 120 preferably is connected to the PIU 110 to spin the coil 122 with pullback (e.g., at least one embodiment of the PIU 110 operates to spin the coil 122 with pullback). The coil 122 delivers torque from a proximal end to a distal end thereof. In one or more embodiments, the coil 122 is fixed with/to the optical probe 124 so that a distal tip of the optical probe 124 also spins to see an omnidirectional view of a biological organ, sample or material being evaluated, such as, but not limited to, hollow organs such as vessels, a heart, etc. For example, fiber optic catheters and endoscopes may reside in the sample arm (such as the sample arm 103 as shown in FIG. 1(A) of an OCT interferometer in order to provide access to internal organs, such as intravascular images, gastro-intestinal tract or any other narrow area, that are difficult to access. As the beam of light through the optical probe 124 inside of the catheter 120 or endoscope is rotated across the surface of interest, cross-sectional images of one or more samples are obtained. In order to acquire three-dimensional data, the optical probe 124 is simultaneously translated longitudinally during the rotational spin resulting in a helical scanning pattern. This translation is most commonly performed by pulling the tip of the probe 124 back towards the proximal end and therefore referred to as a pullback.

In one or more embodiments, the patient user interface 110 may comprise or include a connection component (or interface module), such as a rotary junction, to connect one or more components, such as one or more components of a probe (e.g., a catheter 120 (see e.g., FIGS. 1(A)-1(B)), a needle, a capsule, a patient interface unit (e.g., the patient interface unit 110), etc., to one or more other components, such as, an optical component, a light source (e.g., the light source 101), a deflection section (e.g., the deflection or deflected section 108), the sample arm 102, a motor that operates to power the connection component and/or the patient user interface 110 (e.g., one or more motors may be used to control pullback, to control spin or rotation, etc.), etc. For example, when the connection member or interface module is a rotary junction, the rotary junction may be at least one of: a contact rotary junction, a lensless rotary junction, a lens-based rotary junction, or other rotary junction known to those skilled in the art.

As shown in FIG. 1(B), the catheter 120 may be disposed or positioned in a sheath 121. The sheath 121 may be transparent or semitransparent, may be extruded, and may be single or multilayer. In one or more embodiments, the sheath 121 may be employed with a probe in any application, such as, but not limited to, an OCT needle, an OCT capsule, etc. The catheter 120 is used with the sheath 121 for illustrative purposes, and any probe or catheter may be used with the sheath 121 and/or OCT techniques discussed herein.

The OCT system 100 may be configured as a tomographic image processing apparatus to perform one or more imaging modalities that may include, for example, TD-OCT, spectral domain OCT, FD-OCT, or the like. Since tomographic image processing configurations are generally known, a detailed description will be omitted here.

The OCT system 100 obtains information relating to the sample 106 from radiation reflected back and/or backscattered from regions of the target. In TD-OCT, the reflectivity of the radiation from the sample arm interfere with that coming from the reference arm, whose path is modified within a certain time interval. The displacement of the reference arm can be the measurement of the distance of the target that has caused the reflection. In FD-OCT, the mechanical translation of the reference arm does not occur, and the interference of the light beams oscillates according to the frequency difference.

The image processing apparatus configured as an optical coherence tomography (OCT) system 100 is used to obtain images related to the sample 106,

Intraluminal imaging configurations may display cross sectional images. Intraluminal imaging may acquire high resolution cross-sectional images of tissues or materials, and may enable real time visualization. Intraluminal imaging systems normally sample the physical space in polar coordinates (e.g., radius, r, and angle, θ). Digital image representation is rectangular. Polar coordinates are then obtained after converting from the rectangular to polar representation, to display the OCT images as a lumen cross-section.

Depth range in OCT imaging may be limited by acquisition electronics. In FD-OCT, for example, the depth range can be calculated using the formula Δz=ζₒ²/4nδλ and can be limited by the center wavelength λₒ of the light source, the spectral resolution δλ, and the refractive index n. There is a sensitivity roll-off issue for FD-OCT, which can limit the practical imaging depth range. For the case of spectral domain OCT, the sensitivity roll-off effect is due to the finite pixel number of the line scanning camera. Due to this effect, the visibility of the spectral interference fringe is maximized when the optical path difference is zero and decays as the path length difference increases. Another limitation on the imaging depth of the FD-OCT is posed by the finite depth of focus of the objective lens.

For any given imaging configuration, analog to digital conversion or detector bandwidth is normally a limiting factor. The beam profile may also be a limiting factor and as such for intravascular imaging systems FOV has generally been limited to less than to mm, or a 5 mm imaging depth range.

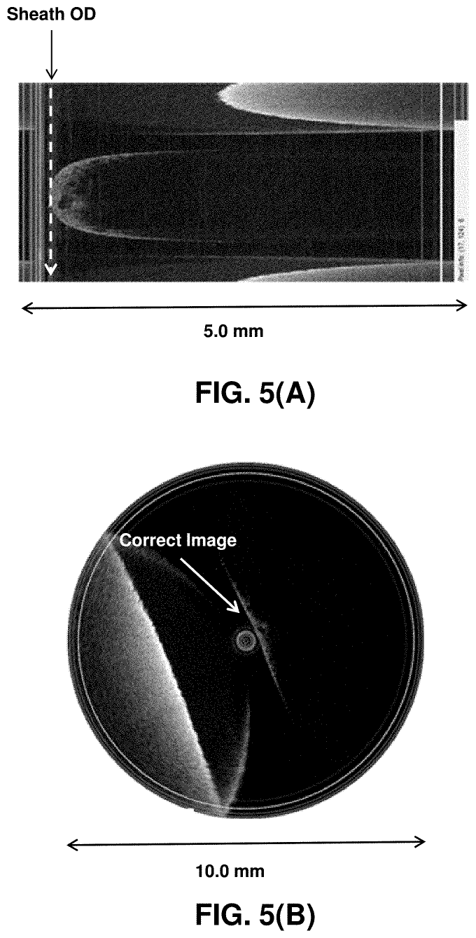

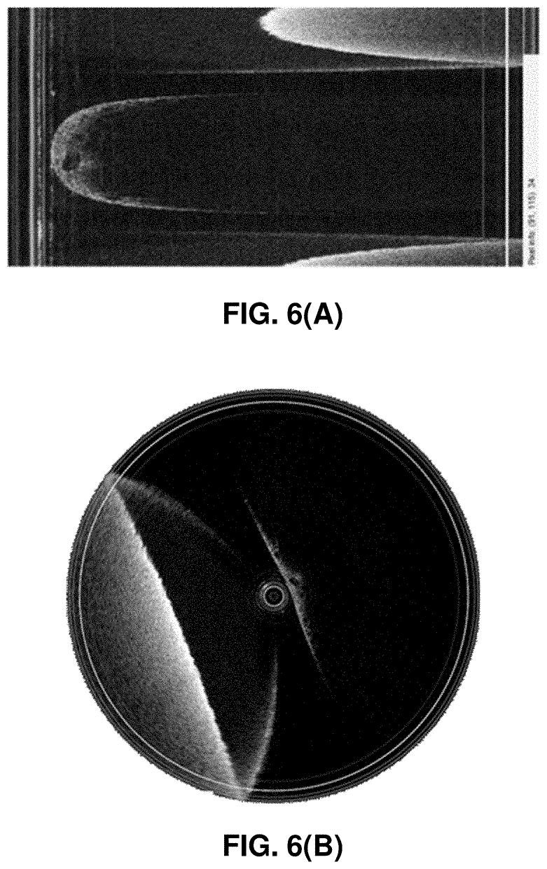

For accurate quantitative dimensional measurement in polar images, pixels mapping of the catheter sheath outer diameter reflection, for example, should represent the actual physical location and size of the sheath outer diameter, otherwise the image will be warped through either expansion or contraction. Other references like an artefactual or intentional reflection from the catheter distal optics or sheath inner diameter can be used instead of the sheath outer diameter reflection as long as the physical location of the reference is known. FIG. 5(B) shows an image with a catheter outer sheath used to accurately represent the physical size of the catheter.

With a maximum FOV, FOV_{MAX}=10 mm, a D_{CATH}=0.9 mm catheter outer diameter, and a minimum L_{T}=1.0 mm tissue penetration, assuming a similar index of refraction (IOR) for catheter vessel and lumen, it can be seen that the largest vessel lumen than can be imaged is (FOV_{MAX}+D_{CATH})/2−L_{T}=4.45 mm. However, when taking for example θₑ=20° tilt angle due to catheter eccentricity and θ_{b}=14° oblique beam illumination, then the maximum vessel lumen that can be imaged is reduced to [Cos (θₑ+θ_{b})*FOV_{MAX}/2]+(D_{CATH}/2)−L_{T}=3.6 mm, thus limiting the size of vessels that can be reliably imaged.

For accurate quantitative dimensional measurement, a portion of the depth range is generally lost to image a portion of the catheter so that the pixel representing the sheath outer diameter reflection is the same as the actual physical location of the sheath outer diameter reflection, which is the object of z-offset calibration.

In aspects of the present disclosure, several image processing steps may be implemented to effectively increase the imaging field of view (FOV).

The image processing system as described herein may obtain reflections from the catheter 120, and adjust so as to place the displayed representation of the catheter sheath 121 a predetermined distance beyond a correct position in a manner where reflections from the catheter 120 do not wrap and/or overlap a desired field of view.

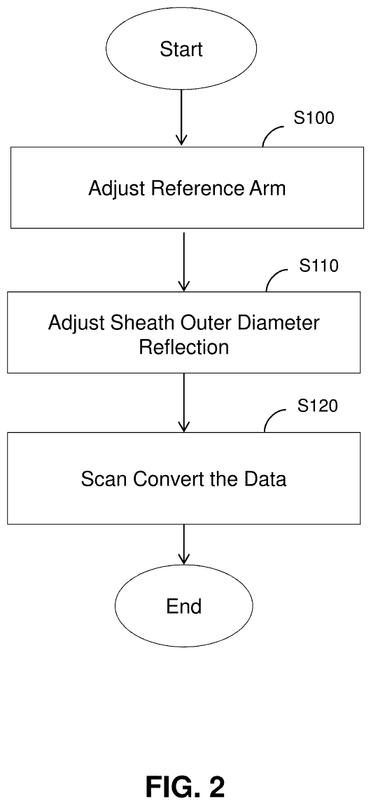

In aspects of the present disclosure, several image processing steps may be implemented to effectively increase the imaging field of view (FOV). FIG. 2 illustrates a flow chart of image processing steps according to a first exemplary embodiment of the present disclosure. The image processing steps of FIG. 2 may be performed during initial calibration of the image processing apparatus.

In step S100, for example, the reference arm 102 of the OCT system 100 may be adjusted so as to place the catheter sheath 121 a predetermined distance beyond the correct position in a manner where reflections from the catheter 120 do not wrap and/or overlap the desired image field. In step S100, the reference reflection adjusted so that catheter reflections wrap about a center of the image but not deeper than the outer diameter reflection. Image object reflections can start anywhere from the catheter outer sheath 121 interface and beyond. The predetermined distance may be based in part on where reflections of the sheath inner diameter wrap around but do not extend beyond the catheter outer diameter reflection. By doing this, improvements in imaging technology may take place, for example, by increasing the imaging field of view (FOV), where vessel sizes larger than 3 mm can be reliably imaged.

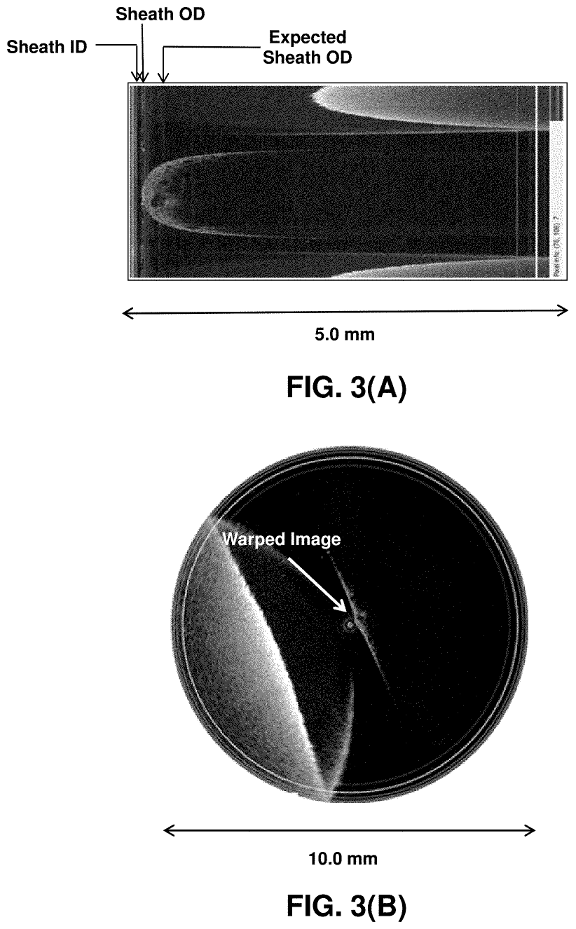

FIG. 3(A) illustrates a digital image representation of an image, and FIG. 3(B) illustrates the digital image of FIG. 3(A) converted to polar representation. In a case where the image is scan converted without further processing, measurements of the image may be incorrect and the image may appear to be warped, as shown in FIG. 3(B).

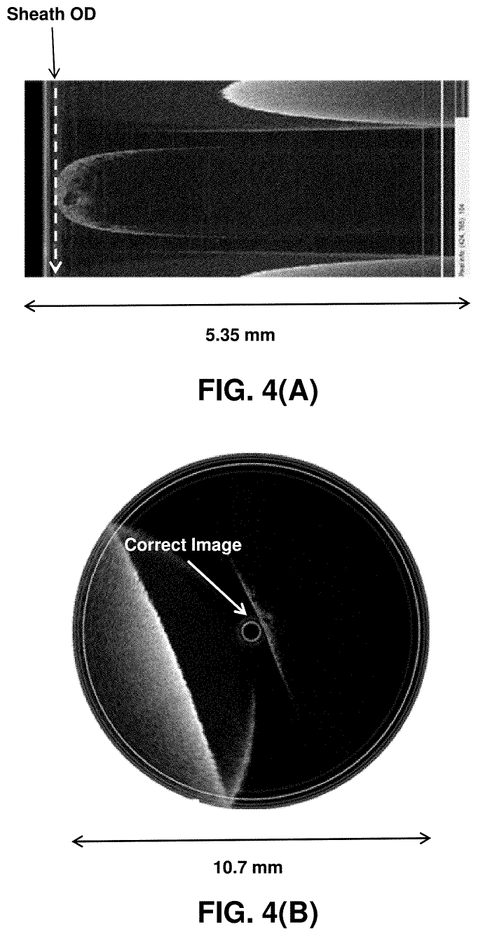

FIG. 4(A) illustrates a digital image representation of an image, and FIG. 4(B) illustrates the digital image of FIG. 4(A) converted to polar representation.

Step S110 may adjust the sheath outer diameter reflection to match the expected sheath outer diameter reflection location. This occurs, not through physical adjustment of the reference arm 102, but rather through padding the image with black pixel data, as shown in FIG. 4(B). In step S110, data is padded in an A-line direction to a position near where the catheter sheath 121 is. The data padding may involve padding pixels at the lowest depth. For example, the initial A-line may be represented by an array with 1,000 elements. This is padded to create an array with 1,100 elements whereas the first too elements contain a value of zero. The padding, in effect, adds an additional number of elements to the A-line image to create image data having all the original data and additional data.

Other data may be used in the image rather than black pixel data including, for example, background pixel data, a cartoon of the catheter 120, other adjunct information about the image, or the like. Alternatively, the data may be compressed so as to maintain a fixed, initial number of pixels. A side effect of compressing the data may occur by degrading sampling resolution. However, this may not be substantial in a case where the data may be down-sampled, for example, by a factor of two or more, in a scan conversion step.

The adjustment of step S100 and/or S110 may be done line by line, frame by frame, or by sector (the average of several A-lines). When the image is padded with blank or background pixel data, etc., this data may be different for each line, frame, or sector (e.g., the number of elements added to each line is defined for that line, frame, or sector. Alternatively, the padding data may be a single, global padding data that is added to each line, frame, or sector (e.g., each line is padded with 100 elements containing a value of zero).

The adjustment of step S100 and/or S110 may be implemented to continuously correct for, for example, any small changes to the sheath location during a pullback. This continual adjustment may be done line by line, frame by frame, or by sector (the average of several A-lines). In some embodiments the adjustment of the reference arm S100 is done less often than the adjustment of the sheath S110.

In step S120, the data is scan converted. In a case where reflections of the sheath inner diameter wrap around but do not extend beyond the catheter outer diameter reflection, accurate measurements may be obtained in a manner where the recovered FOV is larger than a field of view without the above process. In step 120, the data is reconstructed such that the catheter outer diameter reflection accurately reflects the physical size of the catheter 120. The recovered FOV, for example, may be 10.7 mm or about 7% larger than the original FOV. This may lead to an increase in the maximum vessel size that may be reliably imaged, for example, from 3.6 mm to 3.9 mm, or an improvement of more than about 8%. Larger improvements may be obtained for larger diameter catheters. The recovered FOV for this example may be determined through with the following equation [Cos (θₑ+θ_{b})*FOV_{Rec}/2]+(D_{CATH}/2)−L_{T}=3.9 mm.

The number of pixels padded in the A-line direction may be continuously or sporadically updated to account for any minor changes in catheter sheath reflection locations due to small changes in the OCT system due to, for example, temperature gradient changes, stresses to the catheters or other parts of the interferometer along the optical axis.

In the first embodiment, data is padded in an A-line direction to a position near the catheter sheath. In the second embodiment, which is similar to the first embodiment, data is shifted from the opposite direction to where the catheter sheath is, rather than padding data in the A-line direction to a position of the catheter sheath, as in the first embodiment. In other words, this may be considered as cropping data from the deepest section of the image and padding the data at the lowest depth data.

This is advantageous in that the number of pixels in the depth direction may be fixed, thereby facilitating reduction or easing of the computation burden. Possible disadvantages may take place in that there may be no increase in FOV, however, usable FOV may be recovered, for example, in a case where there may be artifacts in the deepest end of the image. For example, this is the case, as illustrated in FIG. 3(B), where the highest depth has artifacts that may be caused by, for example, DC noise and other autocorrelation noise. The brightest deep line is a separate artifact from other parts of the OCT system, and so are the fainter lines that may be shallower than that.

The image processing steps of FIG. 2 may be performed during initial calibration of the image processing apparatus.

In step S100 of the second embodiment of FIG. 2, for example, the reference arm may be adjusted so as to place the catheter sheath a predetermined distance beyond the correct position in a manner where reflections from the catheter do not wrap and/or overlap the desired image field. Image object reflections can start anywhere from the catheter outer sheath interface and beyond. The predetermined distance may be based in part on where reflections of the sheath inner diameter wrap around but do not extend beyond the catheter outer diameter reflection. By doing this, improvements in imaging technology may take place, for example, by increasing the imaging FOV, where vessel sizes larger than 3 mm can be reliably imaged. In a case where the image is scan converted without further processing, measurements of the image may be incorrect and the image may appear to be warped, as shown in FIG. 3(B).

In step S110, the sheath outer diameter reflection may be adjusted to match the expected sheath outer diameter reflection location. This occurs, not through physical adjustment of the reference arm, but rather by shifting the data in an opposite direction to where padding was performed in the first embodiment. The process of shifting the data may involve shifting pixels from the highest depth into the lowest depth. For example, the initial A-line may be represented by an array with 1,000 elements. The whole array elements are circularly shifted in step S110 such that, for example, element 990 becomes element 90 and as such element 1 becomes element 101, element 750 becomes element 850.

In step S120, the data is scan converted. In a case where reflections of the sheath inner diameter wrap around but do not extend beyond the catheter outer diameter reflection, accurate measurements may be obtained in a manner where the recovered FOV is larger than a field of view without the above process. The recovered FOV, for example, may be 10.7 mm or about 7% larger than the original FOV the recovered FOV. This may lead to an increase in the maximum vessel size that may be reliably imaged, for example, from 3.6 mm to 3.9 mm, or an improvement of more than about 8%. Larger improvements may be obtained for larger diameter catheters. The recovered FOV for this example may be determined through with the following equation [Cos (θₑ+θ_{b})*FOV_{Rec}/2]+(D_{CATH}/2)−L_{T}=3.9 mm.

The present disclosure generally achieves improvements in image processing technology, for example, by increasing the size of vessels that can be imaged without changing the acquisition electronics or catheter size. For example, imaging the catheter is not the object of intraluminal imaging but imaging the lumen of the vessel itself and underlying vessel tissue structures, and devices like stents or bioresorbable vascular scaffolds is. As such, pushing catheter reflections out of the desired useable imaging FOV can lead to substantial recovery of FOV for imaging the desired objects rather than keeping a portion of it lost to imaging reflections from the catheter.

The present disclosure generally achieves improvements in image processing technology, for example, by increasing the size of vessels that can be imaged without changing the acquisition electronics or catheter size. Imaging the catheter is not the object of intraluminal imaging but imaging the lumen of the vessel itself and underlying tissue structures, and devices like stents or bioresorbable vascular scaffolds is. As such, pushing catheter reflections out of the desired useable imaging FOV can lead to substantial recovery of FOV for imaging the desired objects rather than keeping a portion of it lost to imaging reflections from the catheter.

There are many ways to implement the imaging processing system 100 including both the control of the OCT system and the image processing by using one or more computer systems. Various components of a computer system 1200 are provided in FIG. 7. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS or other connection lines (e.g., connection line 1213) between one or more of the aforementioned components (e.g., as shown in FIG. 7). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a system, such as the system 100 discussed herein above, via one or more lines 1213), and one or more other computer systems 1200 may include one or more combinations of the other aforementioned components. The CPU 1201 is configured to read and perform computer-executable instructions stored in a storage medium. The computer-executable instructions may include those for the performance of the methods and/or calculations described herein. The system 1200 may include one or more additional processors in addition to CPU 1201, and such processors, including the CPU 1201, may be used, for example, to create the digital representations of imaging data and adjust the reference arm and/or sheath outer diameter reflection. The system 1200 may further include one or more processors connected via a network connection (e.g., via network 1206). The CPU 1201 and any additional processor being used by the system 1200 may be located in the same telecom network or in different telecom networks.

The I/O or communication interface 1205 provides communication interfaces to input and output devices, which may include the light source lot, a spectrometer, a microphone, a communication cable and a network (either wired or wireless), a keyboard 1210, a mouse, a touch screen or screen 1209, a light pen and so on. The Monitor interface or screen 1209 provides communication interfaces thereto.

Any methods and/or data of the present disclosure, such as the methods for adjusting image data to place a catheter sheath a predetermined distance beyond a correct position such that reflections from the catheter do not wrap and/or overlap a desired field of view as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive, SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal. The computer-readable storage medium may include media that store information for predetermined or limited or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, systems, and computer-readable storage mediums related to the processors, such as, but not limited to, the processor of the aforementioned computer 1200, etc., as described above may be achieved utilizing suitable hardware, such as that illustrated in the figures. Functionality of one or more aspects of the present disclosure may be achieved utilizing suitable hardware, such as that illustrated in FIG. 13. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 13) may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components. Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium.

As aforementioned, hardware structure of an alternative embodiment of a computer or console 1200′ is shown in FIG. 8. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. Preferably, the computer or console 1200′ includes a display 1209. The computer 1200′ may connect with the rotary junction, a motion control unit and/or one or more motors via the operation interface 1214 or the network interface 1212. A computer, such as the computer 1200′, may include the MCU in one or more embodiments. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, the computer 1200′, etc.

A computer program is stored in the SSD 1207, and the CPU 1201 loads the program onto the RAM 1203, and executes the instructions in the program to perform one or more processes described herein, as well as the basic input, output, calculation, memory writing and memory reading processes.

The computer, such as the computer 1200, 1200′, may communicate with the PIU 110 (and/or a rotary junction and/or at least one motor being used therewith) and one or more other components of a system, such as the system 100, 100′, 100″, etc. to perform imaging, and constructs or reconstructs an image from the acquired data. The monitor or display 1209 displays the constructed or reconstructed image, and may display other information about the object to be imaged. The monitor 1209 may also provide a graphical user interface for a user to operate an OCT system (e.g., the system 100, the system 100′, the system 100″, etc.). An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs the system (e.g., the system 100, the system 100′, the system 100″, etc.) to set, change, start or end the imaging. The laser source 101 and any other component of the systems discussed herein may have interfaces to communicate with the computers 1200, 1200′ to send and receive status information and the control signals.

The present disclosure and/or one or more components of devices, systems and storage mediums, and/or methods, thereof also may be used in conjunction with any suitable optical assembly or OCT probes including, but not limited to, arrangements and methods for providing multimodality microscopic imaging of one or more biological structure, such as those disclosed in U.S. Pat. Nos. 7,872,759; 8,289,522; and U.S. Pat. No. 8,928,889 to Tearney et al. and arrangements and methods of facilitating photoluminescence imaging, such as those disclosed in U.S. Pat. No. 7,889,348 to Tearney et al., as well as disclosures directed to multimodality imaging disclosed in U.S. Pat. No. 9,332,942 and U.S. Patent Publication Nos. 2009/0192358, 2010/0092389, 2012/0101374 and 2016/0228097, each of which patents and patent publications are incorporated by reference herein in their entireties.

### Other Embodiments

Embodiment(s) of the present disclosure can also be realized by a computerized configuration(s) of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computerized configuration(s) of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computerized configuration(s) may comprise one or more processors (e.g., central processing unit (CPU), micro processing unit (MPU)) and may include a network of separate computers or separate processors to read out and execute the computer executable instructions. The computer executable instructions may be provided to the computerized configuration(s), for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

Although the disclosure herein has been described with reference to particular embodiments, it is to be understood that these embodiments are merely illustrative of the principles and applications of the present disclosure (and are not limited thereto). It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

What is claimed is:

1. An image processing apparatus comprising:
a reference arm;
a sample arm;
a catheter;
a catheter sheath, wherein the catheter is contained within the catheter sheath; and
one or more processors configured to perform steps of:
obtaining reflections from the catheter; and
adjusting so as to place a reflection defining the catheter sheath a predetermined distance beyond a correct position in a manner where reflections from the catheter do not wrap and/or overlap a desired field of view.

2. The image processing apparatus according to claim 1, wherein the adjusting step involves padding pixels at a lowest depth.

3. The image processing apparatus according to claim 2, wherein padding pixels at a lowest depth comprises padding all frames with the same number of pixels.

4. The image processing apparatus according to claim 2, wherein the adjusting step is done continuously during an image acquisition.

5. The image processing apparatus according to claim 1, wherein the adjusting step shifting pixels from the highest depth into the lowest depth.

6. The image processing apparatus according to claim 5, wherein the adjusting step is done continuously during an image acquisition.

7. The imaging processing apparatus according to claim 6, wherein the continuous adjustment is done line-by-line.

8. The image processing apparatus according to claim 1, wherein the adjusting step is implemented during initial calibration of the image processing apparatus.

9. The image processing apparatus according to claim 1, wherein the one or more processors is further configured to adjust the reflection defining the reference arm a predetermined distance beyond a correct position in a manner where reflections from the catheter do not wrap and/or overlap a desired field of view.

10. The image processing apparatus according to claim 1, further comprising a light source.

11. The image processing apparatus according to claim 1, further comprising a splitter.

12. The image processing apparatus according to claim 1, further comprising a reference mirror.

13. The image processing apparatus according to claim 1, further comprising a display.

14. The image processing apparatus according to claim 1, wherein the image processing apparatus implements an imaging modality which is time domain optical coherence tomography, spectral domain optical coherence tomography, or frequency domain optical coherence tomography.

15. The image processing apparatus according to claim 1, wherein the reflections from the catheter are adjusted while in the rectangular representation.

16. An image processing method for an image processing apparatus, the method comprising:
obtaining reflections from a catheter; and
adjusting so as to place a catheter sheath a predetermined distance beyond a correct position in a manner where reflections from the catheter do not wrap and/or overlap a desired field of view.

17. The image processing method according to claim 16, wherein the adjusting involves padding pixels at a lowest depth.

18. The image processing method according to claim 16, wherein the adjusting involves shifting pixels from the highest depth into the lowest depth.

19. The image processing method according to claim 16, wherein the adjusting is implemented during initial calibration of the image processing apparatus.

20. A storage medium storing a program for causing a computer to execute an image processing method for an image processing apparatus, the method comprising:
obtaining reflections from a catheter; and
adjusting so as to place a reflection defining a catheter sheath a predetermined distance beyond a correct position in a manner where reflections from the catheter do not wrap and/or overlap a desired field of view.
